(19) United States
(12) Patent Application Publication
(10) Pub. No.: US 2011/0119792 A1
(43) Pub. Date: May 19, 2011

(54) Genes Controlling Plant Root Growth And Development For Stress Tolerance And Method Of Their Use

(76) Inventors: Henry T. Nguyen, Columbia, MO (US); Babu Valliyodan, Columbia, MO (US)

(21) Appl. No.: 12/966,981

(22) Filed: Dec. 13, 2010

Related U.S. Application Data
(63) Continuation-in-part of application No. 12/138,392, filed on Jun. 12, 2008.
(60) Provisional application No. 61/284,111, filed on Dec. 12, 2009.
(60) Provisional application No. 60/934,321, filed on Jun. 12, 2007.

(51) Int. Cl. C12N 15/82; A01H 5/00

(52) U.S. Cl. 800/290; 800/298; 800/312; 800/320.1; 800/320.2; 800/320.3; 800/314

(57) ABSTRACT

Microarrays are employed to analyze soybean transcriptome under water stress conditions in different regions of the root at vegetative stage. Drought responsive genes and transcription factors are identified which may be used for enhancing drought tolerance in soybean or other plants through genetic/metabolic engineering. This disclosure pertains to nucleic acid molecules isolated from soybean and maize that encode polypeptides that may be important for drought tolerance. The disclosure also relates to methods of using these genes from soybean in transgenic plants to confer the desired agronomic traits, and to use such genes or regulatory elements thereof to assist germplasm enhancement by molecular breeding or to identify other factors or chemicals that may enhance a plant's capability to grow under drought conditions.

### RELATED APPLICATIONS

This application claims priority to U.S. Provisional Application No. 61/284,111 filed on Dec. 12, 2009. This application is also a continuation-in-part of U.S. patent application Ser. No. 12/138,392 filed on Jun. 12, 2008, which claims priority of U.S. Provisional Application No. 60/934,321 filed on Jun. 12, 2007. The contents of all applications mentioned above are hereby incorporated into this application by reference.

### BACKGROUND

1. Field of the Invention

The present invention relates to methods and materials for identifying genes and the regulatory network in a plant that control the plant's response to various environmental conditions and/or stress. More particularly, the present invention relates to genes in the root of a plant that help confer drought resistance to the plant.

2. Description of the Related Art

Drought is one of the major abiotic stress factors limiting crop productivity worldwide. Global climate changes may further exacerbate the drought situation in major crop-producing countries. Although irrigation may in theory solve the drought problem, it is usually not a viable option because of the cost associated with building and maintaining an effective irrigation system, as well as other non-economical issues, such as the general availability of water (Boyer, 1983). Thus, alternative means for alleviating plant water stress are needed.

In soybean, drought stress during flowering and early pod development significantly increases the rate of flower and pod abortion, thus decreasing final yield (Boyer 1983; Westgate and Peterson 1993). Soybean yield reduction of 40% because of drought is common experience among soybean producers in the United States (Muchow & Sinclair, 1986; Specht et al. 1999).

Mechanisms for selecting drought tolerant plants fall into three general categories. The first is called drought escape, in which selection is aimed at those developmental and maturation traits that match seasonal water availability with crop needs. The second is dehydration avoidance, in which selection is focused on traits that: lessen evaporatory water loss from plant surfaces or maintain water uptake during drought via a deeper and more extensive root system. The last mechanism is dehydration tolerance, in which selection is directed at maintaining cell turgor or enhancing cellular constituents that protect cytoplasmic proteins and membranes from drying.

The molecular mechanisms of abiotic stress responses and the genetic regulatory networks of drought stress tolerance have been reviewed recently (Neumann 2008; Wang et at 2003; Vinocur and Altman 2005; Chaves and Oliveira 2004; Shinozaki et al. 2003). Plant modification for enhanced drought tolerance is mostly based on the manipulation of either transcription and/or signaling factors or genes that directly protect plant cells against water deficit. Despite much progress in the field, understanding the basic biochemical and molecular mechanisms for drought stress perception, transduction, response and tolerance remains a major challenge in the filed. Utilization of the knowledge on drought tolerance to generate plants that can tolerate extreme water deficit condition is a even bigger challenge.

Analysis of changes in gene expression within a target plant is important for revealing the transcriptional regulatory networks. Elucidation of these complex regulatory networks may contribute to our understanding of the responses mounted by a plant to various stresses and developmental changes, which may ultimately lead to crop improvement. DNA microarray assays (Schena et al. 1995; Shalon et al. 1996) have provided an unprecedented opportunity for the generation of gene expression data on a whole-genome scale.

Gene expression profiling using cDNAs or oligonucleotides microarray technology has advanced our understanding of gene regulatory network when a plant is subject to various stresses (Bray 2004; Denby and Gehring 2005). For example, numerous genes that respond to dehydration stress have been identified in Arabidopsis and have been categorized as “rd” (responsive to dehydration) or “erd” (early response to dehydration) (Shinozaki and Yamaguchi-Shinozaki 1999).

There are at least four independent regulatory pathways for gene expression in response to water stress. Out of the four pathways, two are abscisic acid (ABA) dependent and the other two are ABA independent (Shinozaki and Yamaguchi-Shinozaki 2000). In the ABA independent regulatory pathways, a cis-acting element is involved and the Dehydration-responsive element/C-repeat (DRE/CRT) has been identified. DRE/CRT also functions in cold response and high-salt-responsive gene expression. When the DRE/CRT binding protein DREB1/ICBF is overexpressed in a transgenic Arabidopsis plant, changes in expression of more than 40 stress-inducible genes can be observed, which lead to enhanced tolerance to freeze, high salt, and drought (Seki et al, 2001; Fowler and Thomashow 2002; Murayama et al. 2004).

In summary, the production of microarrays and the global transcript profiling of plants have revolutionized the study of gene expression which provides a unique snapshot of how these plants are responding to a particular stress. However, no transcriptional profiling or transcriptome changes have been reported for soybean plants under water stress conditions. A well designed analysis of gene expression in soybean grown under drought may help illuminate the regulatory networks in soybean under these adverse conditions, which may further lead to development of new soybean plant that can better tolerate drought conditions than conventional strains.

### SUMMARY

The instrumentalities described herein overcome the problems outlined above and advance the art by providing genes and DNA regulatory elements in plant roots which may play an important part in response to drought conditions. These genes are collectively called Root Specific Drought Response Genes (RSDRGs) in this disclosure. Methodology is also provided whereby these drought responsive genes may be introduced into a plant to enhance its capability to grow and reproduce under water deficit conditions. The RSDRGs or fragments thereof may be introduced and expressed in the roots of a host plant, or alternatively, they may be expressed in other organs of the host plants. Regulatory elements, such as promoters and enhancers of the RSDRGs, may also be employed to control expression of genes that are not yet identified but may prove beneficial for enhancing a plant's capability to grow under drought conditions.

In crop plants, root growth is often less sensitive to low tissue water potential than shoot growth. This root specific trait confers drought tolerance to some plants to certain degree, but the mechanisms underlying root growth and development under drought stress conditions (low water potential) are not well understood. To discover genes and to identify mechanisms that determine these responses to low water potential conditions, gene expression patterns were profiled from three regions of water-stressed and well-watered roots, respectively. These analyses, along with comparisons with the gene expression patterns in maize root regions under low water potentials with those of soybean, support the notion that there is a distinct and species-specific pattern of gene expression under water deficit conditions in root tissues.

This disclosure provides novel drought responsive genes that may play a role in root growth and development under normal conditions. These candidates may be used for enhanced drought tolerance in soybean and other crops through genetic/metabolic engineering. The genes and methods disclosed herein apply not only to soybean and maize but also to drought stress tolerance/avoidance mechanisms in other plants. This disclosure provides both early and late stage stress responsive genes/proteins controlling root growth and development under water deficits. One or more of these genes or their fragments from soybean or maize as disclosed herein may be expressed in transgenic plants to confer certain agronomic traits to a host plant. The genes may also be used to assist germplasm enhancement by molecular breeding.

More particularly, microarray experiments are conducted to analyze the gene expression pattern in different root regions of soybean plants in response to drought stress. Tissue specific transcriptomes may be compared to help elucidate the transcriptional regulatory network and facilitate the identification of stress specific genes and promoters. For purpose of this disclosure, genes whose expression are either up- or down-regulated in response to drought condition are referred to as Drought Response Genes (or DRGs). Some DRGs may show tissue specific expression patterns in response to drought condition. Those DRGs whose expression are up- or down-regulated in the roots are also referred to as Root Specific Drought Response Genes (“RSDRGs”). For purpose of this disclosure, a “RSDRG protein” refers to a protein encoded by a RSDRG.

The microarray experiments described in this disclosure may not have uncovered all the RSDRGs in all plants, or even in soybean alone, due to the variations in experimental conditions, and more importantly, due to the different gene expressions among different plant species. It is also to be understood that certain RSDRGs disclosed here may have been identified and studied previously; however, regulation of their expression under drought condition or their role in drought response may not have been appreciated in previous studies. Alternatively, some RSDRGs may contain novel coding sequences. Thus, it is an object of the present disclosure to identify known or unknown genes whose expression levels are altered in response to drought condition.

For purpose of this disclosure, if a transgenic plant or a genetically altered plant is able to grow at a similar rate and reproduce at a similar yield when water is limited as compared to the host plant or the parental plant with sufficient water supply, it can be said that the transgenic plant or the genetically altered plant has gained drought resistance (or tolerance), or in other words, that such plant is more drought resistant, or more tolerant to drought (or water deficit) conditions than the host or the parent. In one aspect, the transgenic or altered plant may be able to grow at a similar rate and reproduce at a similar yield when water available to the plant is 20% less, 30% less, 40% less, 50% less, more preferably 60% less, than the water available to the host or parental plant. In order to generate a transgenic plant that is more tolerant to drought condition when compared to a host plant, the expression levels of a protein encoded by an endogenous Root Specific Drought Response Gene (RSDRG) or a fragment thereof may be altered to confer a drought resistant phenotype to the host plant.

For example, the transcription, translation or protein stability of the protein encoded by the RSDRG may be modified so that the levels of this protein are rendered significantly higher than the levels of this protein would otherwise be even under the same drought condition. To this end, either the coding or non-coding regions, or both, of the endogenous RSDRG may be modified.

In another aspect, in order to generate a transgenic plant that is more tolerant to drought condition when compared to a host plant, the method may comprise the steps of: (a) introducing into a plant cell a construct comprising a Root Specific Drought Response Gene (RSDRG) or a fragment thereof encoding a polypeptide; and (b) generating a transgenic plant expressing said polypeptide or a fragment thereof. In one embodiment, the Drought Response Gene or a fragment thereof is derived from a plant that is genetically different from the host plant. In another embodiment, the Drought Response Gene or a fragment thereof is derived from a plant that belongs to the same species as the host plant. For instance, an RSDRG identified in soybean may be introduced into soybean as a transgene to confer upon the host increased capability to grow and/or reproduced under mild to severe drought conditions.

The RSDRGs disclosed here include known genes as well as genes whose functions are not yet fully understood. Nevertheless, both known or unknown RSDRGs may be placed under control of a promoter and be transformed into a host plant in accordance with standard plant transformation protocols. The transgenic plants thus obtained may be tested for the expression of the RSDRGs and their capability to grow and/or reproduce under drought conditions as compared to the original host (or parental) plant.

Although the RSDRGs disclosed herein are identified in soybean, they may be introduced into other plants as transgenes. Examples of such other plants may include but are not limited to corn, wheat, rice, and cotton. In another aspect, homologs in other plant species may be identified by PCR, hybridization or by genome search which may share substantial sequence similarity with the RSDRGs disclosed herein. In a preferred embodiment, such a homolog shares at least 90%, more preferably 98%, or even more preferably 99% sequence identity with a protein encoded by a soybean RSDRG.

It is further an object to identify the non-coding regulatory sequences of the RSDRGs which are calleed Root Specific Drought Response Regulatory Elements (RSDRREs) for purpose of this disclosure. These RSDRREs may be used to prepare DNA constructs for the expression of genes of interest in a host plant. The RSDRREs or the RSDRGs may also be used to screen for factors or chemicals that may affect the expression of certain RSDRGs by interacting with a RSDRRE. Such factors or chemicals may be used to induce drought responses by activating expression of certain genes in a plant.

In one aspect, the genes of interest may be genes from the transgenic host plant, or they may be from other plants or even from non-plant organisms. The genes of interest may be those identified and listed in this disclosure, or they may be the homologs of these genes.

In one embodiment, the genes of interest (RSDRGs) may be placed under control of the RSDRREs such that their expression may be upregulated under drought condition. This arrangement is particularly useful for those genes of interest that may not be desirable under normal condition, because such genes may be placed under a tightly regulated RSDRRE which only drives the expression of the genes of interest when water deficit condition is sensed by the plant. Under control of such a RSDRRE, expression of the gene of interest may be only detected under drought condition. The RSDRRE can thus be used to regulate a heterologous gene such that expression of the heterologous gene is induced under drought conditions.

It is an object of this disclosure to provide a system and a method for the genetic modification of a plant, to increase the resistance of the plant to adverse conditions such as drought and/or excessive temperatures, compared to an unmodified plant.

It is another object of the present invention to provide a transgenic plant that exhibits increased resistance to adverse conditions such as drought and/or excessive temperatures as compared to an unmodified plant.

It is another object of the present invention to provide a system and method of modifying a plant, to alter the metabolism or development of the plant.

In one embodiment, a gene of interest may be placed under control of a tissue specific promoter such that such gene of interest may be expressed in specific site, for example, the guard cells. The expression of the introduced genes may enhance the capacity of a plant to modulate guard cell activity in response to water stress. For instance, the transgene may help reduce stomatal water loss. In addition, other characteristics such as early maturation of plants may be introduced into plants to help cope with drought condition.

Preferably, the transgene is under control of a promoter, which may be a constitutive or inducible promoter. An inducible promoter is inactive under normal condition, and is activated under certain conditions to drive the expression of the gene under its control. Conditions that may activate a promoter include but are not limited to light, heat, certain nutrients or chemicals, and water conditions. A promoter that is activated under water deficit condition is preferred.

In another aspect, a tissue specific promoter, an organ specific promoter, or a cell-specific promoter may be employed to control the transgene. Despite their different names, these promoters are similar in that they are only activated in certain cell, tissue or organ types. It is to be understood that a gene under control of an inducible promoter, or a promoter specific for certain cells, tissues or organs may have low level of expression even under conditions that are not supposed to activate the promoter, a phenomenon known as “leaky expression” in the field. A promoter can be both inducible and tissue specific. By way of example, a transgene may be placed under control of a guard cell specific promoter such that the gene can be inducibly expressed in the guard cell of the transgenic plant.

In another aspect, the present disclosure provides a method of generating a transgenic plant having an altered stress response or an altered phenotype compared to an unmodified plant. The coding sequences of the genes that are disclosed to be upregulated may be placed under a promoter such that the genes can be expressed in the transgenic plant. The method may contain two steps: (a) introducing into a plant cell capable of being transformed and regenerated into a whole plant a construct comprising, in addition to the DNA sequences required for transformation and selection in plants, an expression construct including the coding sequence of a gene that a operatively linked to a promoter for expressing said DNA sequence; and (b) recovery of a plant which contains the expression construct.

The transgenic plant generated by the methods disclosed above may exhibit an altered trait or stress response. The altered traits may include increased tolerance to extreme temperature, such as heat or cold; or increased tolerance to extreme water condition such as drought or excessive water. The transgenic plant may exhibit one or more altered phenotype that may contribute to the resistance to drought condition. These phenotypes may include, by way of example, early maturation, increased growth rate, increased biomass, or increased lipid content.

In accordance with the disclosed methods, the coding sequence to be introduced in the transgenic plant preferably encodes a peptide having at least 70%, more preferably at least 90%, more preferably at least 98% identity, and even more preferably at least 99% identity to the polypeptide encoded by the RSDRGs disclosed in this application. In an alternative aspect, DNA sequence may be oriented in an antisense direction relative to said promoter within said construct.

In accordance with the methods of the present invention, the promoter is preferably selected from the group consisting of an constitutive promoter, an inducible promoter, a tissue specific promoter, and organ specific promoter, a cell-specific promoter. More preferably the promoter is an inducible promoter for expressing said DNA sequence under water deficit conditions.

In another aspect, the present invention provides a method of identifying whether a plant that has been successfully transformed with a construct, characterized in that the method comprises the steps of: (a) introducing into plant cells capable of being transformed and regenerated into whole plants a construct comprising, in addition to the DNA sequences required for transformation and selection in plants, an expression construct that includes a DNA sequence selected from at least one of the RSDRGs disclosed herein, said DNA sequence may be operatively linked to a promoter for expressing said DNA sequence; (b) regenerating the plant cells into whole plants; and (c) subjecting the plants to a screening process to differentiate between transformed plants and non-transformed plants.

The screening process may involve subjecting the plants to environmental conditions suitable to kill non-transformed plants, retain viability in transformed plants. For instance by growing the plants in a medium or soil that contains certain chemicals, such that only those plants expressing the transgenes can survive. In one particular embodiment, after obtaining a transgenic plant that appear to be expressing the transgene, a functional screening may be carried out by growing the plants under water deficit conditions to select for those that can tolerate such a condition.

In another aspect, the present disclosure provides a kit for generating a transgenic plant having an altered stress response or an altered phenotype compared to an unmodified plant, characterized in that the kit comprises: an expression construct including a DNA sequence selected from at least one of the RSDRGs disclosed herein, said DNA sequence may be operatively linked to an promoter suitable for expressing said DNA sequence in a plant cell.

Preferably the kit further includes targeting means for targeting the activity of the protein expressed from the construct to certain tissues or cells of the plant. Preferably the targeting means comprises an inducible, tissue-specific promoter for specific expression of the DNA sequence within certain tissues of the plant. Alternatively the targeting means may be a signal sequence encoded by said expression construct and may contain a series of amino acids covalently linked to the expressed protein.

In accordance with the kit of the present invention, the DNA sequence may encode a peptide having at least 70%, more preferably at least 90%, more preferably at least 98%, or even 99% identity to the peptide encoded by coding sequences selected from at least one of the RSDRGs disclosed herein. In one aspect, said DNA sequence may be oriented in an antisense direction relative to said promoter within said construct.

### BRIEF DESCRIPTION OF THE DRAWINGS

FIG. 1 illustrates a whole soybean seedling (A) and the root tip regions of the soybean seedling root (B).

FIG. 2 shows the numbers of genes that show differential expression at the same time point 5 hour (A) or 48 hour (B) between different root regions.

FIG. 3 shows the number of genes within the same root region, R1(A) or R2 (B), that are differentially expressed between two time points 5 hr and 48 hr after the plants have been subjected to water deficit conditions.

FIG. 4 shows the number of genes that are only differentially expressed in R1, R2 or both R1 and R2 regions of the root up to 48 hours after the plants are exposed to drought conditions (upper panel), and the number of genes that show differential expression at 5 hours, 48 hours after being exposed to drought conditions, as well as those that show differential expression at both 5 hours and 48 hours (lower panel).

FIG. 5 shows the number genes that are differentially expressed under drought condition only in the R2 region but not in the R3 region.

FIG. 6 presents pie charts illustrating the different functional categories of genes that show differential expression in the R1 (8A and 8B) or R2 (8C and 8D) regions after having been subjected to water deficit conditions for 5 hour (8A and 8C) or 48 hours (8B and 8D).

FIG. 7 shows the different categories of the “root region and stress specific transcripts” (RRSST) by their known or predicted function.

### DETAILED DESCRIPTION

The methods and materials described herein relate to gene expression profiling using microarrays, and follow-up analysis to decode the regulatory network that controls a plant's response to drought conditions. More particularly, drought response is analyzed at the molecular level to identify root specific genes and/or promoters which may be activated under water deficit conditions. The coding sequences of such genes may be introduced into a host plant to obtain transgenic plants that are more tolerant to drought than unmodified plants.

It is to be understood that the materials and methods are taught by way of example, and not by limitation. The disclosed instrumentalities may be broader than the particular methods and materials described herein, which may vary within the skill of the art. It is also to be understood that the terminology used herein is for the purpose of describing particular embodiments only, and is not intended to be limiting. Further, unless defined otherwise, all technical and scientific terms used herein have the same meaning as commonly understood by one of ordinary skill in the related art. The following terminology and grammatical variants are used in accordance with the definitions set out below.

The present disclosure provides genes whose expression levels are altered in response to stress conditions in soybean plants using genome-wide microarray (or gene chip) analysis of soybean plants grown under water deficit conditions. Those genes identified using microarray analysis may be subject to validation to confirm that their expression levels are altered under the stress conditions. Validation may be conducted using high throughput two-step qRT-PCR and by the delta delta CT method.

Sequences of those genes that have been validated may be subject to further sequence analysis by comparing their sequences to published sequences of various families of genes or proteins. For instance, some of these RSDRGs may encode proteins with substantial sequence similarity to known transcription factors. These transcription factors may play a role in the stress response by activating the transcription of other genes.

The present disclosure provides a system and a method for expressing a protein that may enhance a host's capability to grow or to survive in an adverse environment characterized by water deficit. Although plants are the most preferred host for purpose of this disclosure, the genetic constructs described herein may be introduced into other eukaryotic organisms, if the traits conferred upon these organisms by the constructs are desirable.

The term “genetically altered plant” or “genetically modified plant” refers to a plant whose genetic make-up has been altered or modified such that the modified plant expresses one or more protein that is not normally expressed by the unmodified plant or is expressed at different time or different tissue of the unmodified plant.

The term “transgenic plant” refers to a host plant into which a gene construct has been introduced. A gene construct, also referred to as a construct, an expression construct, or a DNA construct, generally contains as its components at least a coding sequence and a regulatory sequence. A gene construct typically contains at least on component that is foreign to the host plant. For purpose of this disclosure, all components of a gene construct may be from the host plant, but these components are not arranged in the host in the same manner as they are in the gene construct. A regulatory sequence is a non-coding sequence that typically contribute to the regulation of gene expression, at the transcription or translation levels. It is to be understood that certain segments in the coding sequence may be translated but may be later removed from the functional protein. An example of these segments is the so-called signal peptide, which may facilitate the maturation or localization of the translated protein, but is typically removed once the protein reaches its destination. Examples of a regulatory sequence include but are not limited to a promoter, an enhancer, and certain post-transcriptional regulatory elements.

After its introduction into a host plant, a gene construct may exist separately from the host chromosomes. Preferably, the entire gene construct, or at least part of it, is integrated onto a host chromosome. The integration may be mediated by a recombination event, which may be homologous, or non-homologous recombination. The term “express” or “expression” refers to production of RNAs using DNAs as template through transcription or translation of proteins from RNAs or the combination of both transcription and translation.

A “host cell,” as used herein, refers to a prokaryotic or eukaryotic cell that contains heterologous DNA which has been introduced into the cell by any means, e.g., electroporation, calcium phosphate precipitation, microinjection, transformation, viral infection, and/or the like. A “host plant” is a plant into which a transgene is to be introduced. A “parental plant” is the original plant into which genetic changes are to be introduced in order to create a genetically altered plant.

A “vector” is a composition for facilitating introduction, replication and/or expression of a selected nucleic acid in a cell. Vectors include, for example, plasmids, cosmids, viruses, yeast artificial chromosomes (YACs), etc. A “vector nucleic acid” is a nucleic acid vector into which heterologous nucleic acid is optionally inserted and which can then be introduced into an appropriate host cell. Vectors preferably have one or more origins of replication, and one or more sites into which the recombinant DNA can be inserted. Vectors often have convenient markers by which cells with vectors can be selected from those without. By way of example, a vector may encode a drug resistance gene to facilitate selection of cells that are transformed with the vector. Common vectors include plasmids, phages and other viruses, and “artificial chromosomes.” “Expression vectors” are vectors that comprise elements that provide for or facilitate transcription of nucleic acids which are cloned into the vectors. Such elements may include, for example, promoters and/or enhancers operably coupled to a nucleic acid of interest.

“Plasmids” generally are designated herein by a lower case “p” preceded and/or followed by capital letters and/or numbers, in accordance with standard nomenclatures that are familiar to those of skill in the art. Starting plasmids disclosed herein are either commercially available, publicly available on an unrestricted basis, or can be constructed from available plasmids by routine application of well known, published procedures. Many plasmids and other cloning and expression vectors are well known and readily available to those of skill in the art. Moreover, those of skill readily may construct any number of other plasmids suitable for use as described below. The properties, construction and use of such plasmids, as well as other vectors, is readily apparent to those of ordinary skill upon reading the present disclosure.

When a molecule is identified in or can be isolated from a organism, it can be said that such a molecule is derived from said organism. When two organisms have significant difference in the genetic materials in their respective genomes, these two organisms can be said to be genetically different. For purpose of this disclosure, the term “plant” means a whole plant, a seed, or any organ or tissue of a plant that may potentially develop into a whole plant.

The term “isolated” means that the material is removed from its original environment, such as the native or natural environment if the material is naturally occurring. For example, a naturally-occurring nucleic acid, polypeptide, or cell present in a living animal is not isolated, but the same polynucleotide, polypeptide, or cell separated from some or all of the coexisting materials in the natural system, is isolated, even if subsequently reintroduced into the natural system. Such nucleic acids can be part of a vector and/or such nucleic acids or polypeptides could be part of a composition, and still be isolated in that such vector or composition is not part of its natural environment.

A “recombinant nucleic acid” is one that is made by recombining nucleic acids, e.g., during cloning, DNA evolution or other procedures. A “recombinant polypeptide” is a polypeptide which is produced by expression of a recombinant nucleic acid. An “amino acid sequence” is a polymer of amino acid residues (a protein, polypeptide, etc.) or a character string representing an amino acid polymer, depending on context. Either the given nucleic acid or the complementary nucleic acid can be determined from any specified polynucleotide sequence.

The terms “nucleic acid,” or “polynucleotide” refer to a deoxyribonucleotide, in the case of DNA, or ribonucleotide in the case of RNA polymer in either single- or double-stranded form, and unless otherwise specified, encompasses known analogues of natural nucleotides that can be incorporated into nucleic acids in a manner similar to naturally occurring nucleotides. A “polynucleotide sequence” is a nucleic acid which is a polymer of nucleotides (A,C,T,U,G, etc. or naturally occurring or artificial nucleotide analogues) or a character string representing a nucleic acid, depending on context. Either the given nucleic acid or the complementary nucleic acid can be determined from any specified polynucleotide sequence.

A “subsequence” or “fragment” is any portion of an entire sequence of a DNA, an RNA or a polypeptide (also referred to as “a protein”) molecule, up to and including the full sequence. Typically a subsequence or fragment comprises less than the full-length sequence, and is sometimes referred to as the “truncated version.”

Nucleic acids and/or nucleic acid sequences are “homologous” when they are derived, naturally or artificially, from a common ancestral nucleic acid or nucleic acid sequence. Proteins and/or protein sequences are homologous when their encoding DNAs are derived, naturally or artificially, from a common ancestral nucleic acid or nucleic acid sequence. Similarly, nucleic acids and/or nucleic acid sequences are homologous when they are derived, naturally or artificially, from a common ancestral nucleic acid or nucleic acid sequence. The homologous molecules can be termed homologs. For example, any naturally occurring RSDRGs, as described herein, can be modified by any available mutagenesis method. When expressed, this mutagenized nucleic acid encodes a polypeptide that is homologous to the protein encoded by the original RSDRGs. Homology is generally inferred from sequence identity between two or more nucleic acids or proteins (or sequences thereof). The precise percentage of identity between sequences that is useful in establishing homology varies with the nucleic acid and protein at issue, but as little as 25% sequence identity is routinely used to establish homology. Higher levels of sequence identity, e.g., 30%, 40%, 50%, 60%, 70%, 80%, 90%, 95% or 99% or more can also be used to establish homology. Methods for determining sequence identity percentages (e.g., BLASTP and BLASTN using default parameters) are described herein and are generally available.

The terms “identical” or “sequence identity” in the context of two nucleic acid sequences or amino acid sequences of polypeptides refers to the residues in the two sequences which are the same when aligned for maximum correspondence over a specified comparison window. A “comparison window”, as used herein, refers to a segment of at least about 20 contiguous positions, usually about 50 to about 200, more usually about 100 to about 150 in which a sequence may be compared to a reference sequence of the same number of contiguous positions after the two sequences are aligned optimally. Methods of alignment of sequences for comparison are well-known in the art. Optimal alignment of sequences for comparison may be conducted by the local homology algorithm of Smith and Waterman (1981) Adv. Appl. Math. 2:482; by the alignment algorithm of Needleman and Wunsch (1970) J. Mol. Biol. 48:443; by the search for similarity method of Pearson and Lipman (1988) Proc. Nat. Acad. Sci. U.S.A. 85:2444; by computerized implementations of these algorithms (including, but not limited to CLUSTAL in the PC/Gene program by Intelligentics, Mountain View Calif., GAP, BESTFIT, BLAST, FASTA, and TFASTA in the Wisconsin Genetics Software Package, Genetics Computer Group (GCG), 575 Science Dr., Madison, Wis., U.S.A.); the CLUSTAL program is well described by Higgins and Sharp (1988) Gene 73:237-244 and Higgins and Sharp (1989) CABIOS 5:151-153; Corpet et al. (1988) Nucleic Acids Res. 16:10881-10890; Huang et al (1992) Computer Applications in the Biosciences 8:155-165; and Pearson et al. (1994) Methods in Molecular Biology 24:307-331. Alignment is also often performed by inspection and manual alignment.

In one class of embodiments, the polypeptides herein are at least 70%, generally at least 75%, optionally at least 80%, 85%, 90%, 98% or 99% or more identical to a reference polypeptide, e.g., those that are encoded by DNA sequences as set forth by any one of the RSDRGs disclosed herein or a fragment thereof, e.g., as measured by BLASTP (or CLUSTAL, or any other available alignment software) using default parameters. Similarly, nucleic acids can also be described with reference to a starting nucleic acid, e.g., they can be 50%, 60%, 70%, 75%, 80%, 85%, 90%, 98%, 99% or more identical to a reference nucleic acid, e.g., those that are set forth by any one of the RSDRGs disclosed herein or a fragment thereof, e.g., as measured by BLASTN (or CLUSTAL, or any other available alignment software) using default parameters. When one molecule is said to have certain percentage of sequence identity with a larger molecule, it means that when the two molecules are optimally aligned, said percentage of residues in the smaller molecule finds a match residue in the larger molecule in accordance with the order by which the two molecules are optimally aligned.

The term “substantially identical” as applied to nucleic acid or amino acid sequences means that a nucleic acid or amino acid sequence comprises a sequence that has at least 90% sequence identity or more, preferably at least 95%, more preferably at least 98% and most preferably at least 99%, compared to a reference sequence using the programs described above (preferably BLAST) using standard parameters. For example, the BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89:10915 (1989)). Percentage of sequence identity is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

The term “polypeptide” is used interchangeably with the terms “polypeptides” and “protein(s)”, and refers to a polymer of amino acid residues. A ‘mature protein’ is a protein which is full-length and which, optionally, includes glycosylation or other modifications typical for the protein in a given cellular environment.

The term “variant” or “mutant” with respect to a polypeptide refers to an amino acid sequence that is altered by one or more amino acids with respect to a reference sequence. The variant may have “conservative” changes, wherein a substituted amino acid has similar structural or chemical properties, e.g., replacement of leucine with isoleucine. Alternatively, a variant may have “nonconservative” changes, e.g., replacement of a glycine with a tryptophan. Analogous minor variation can also include amino acid deletion or insertion, or both. Guidance in determining which amino acid residues can be substituted, inserted, or deleted without eliminating biological or immunological activity can be found using computer programs well known in the art, for example, DNASTAR software.

A variety of additional terms are defined or otherwise characterized herein. In practicing the instrumentalities described herein, many conventional techniques in molecular biology, microbiology, and recombinant DNA are optionally used. These techniques are well known to those of ordinary skill in the art. For example, one skilled in the art would be familiar with techniques for in vitro amplification methods, including the polymerase chain reaction (PCR), for the production of the homologous nucleic acids described herein.

In addition, commercially available kits may facilitate the purification of plasmids or other relevant nucleic acids from cells. See, for example, EasyPrep™ and FlexiPrep™ kits, both from Pharmacia Biotech; StrataClean™ from Stratagene; and, QIAprep™ from Qiagen. Any isolated and/or purified nucleic acid can be further manipulated to produce other nucleic acids, used to transfect cells, incorporated into related vectors to infect organisms, or the like. Typical cloning vectors contain transcription terminators, transcription initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Vectors are suitable for replication and integration in prokaryotes, eukaryotes, or both.

Various types of mutagenesis are optionally used to modify RSDRGs and their encoded polypeptides, as described herein, to produce conservative or non-conservative variants. Any available mutagenesis procedure can be used. Such mutagenesis procedures optionally include selection of mutant nucleic acids and polypeptides for one or more activity of interest. Procedures that can be used include, but are not limited to: site-directed point mutagenesis, random point mutagenesis, in vitro or in vivo homologous recombination (DNA shuffling), mutagenesis using uracil-containing templates, oligonucleotide-directed mutagenesis, phosphorothioate-modified DNA mutagenesis, mutagenesis using gapped duplex DNA, point mismatch repair, mutagenesis using repair-deficient host strains, restriction-selection and restriction-purification, deletion mutagenesis, mutagenesis by total gene synthesis, double-strand break repair, mutagenesis by chimeric constructs, and many others known to persons of skill in the art.

In one embodiment, mutagenesis can be guided by known information about the naturally occurring molecule or altered or mutated naturally occurring molecule. By way of example, this known information may include sequence, sequence comparisons, physical properties, crystal structure and the like. In another class of mutagenesis, modification is essentially random, e.g., as in classical DNA shuffling.

Polypeptides may include variants, in which the amino acid sequence has at least 70% identity, preferably at least 80% identity, typically 90% identity, preferably at least 95% identity, more preferably at least 98% identity and most preferably at least 99% identity, to the amino acid sequences as encoded by the DNA sequences set forth in any one of the RSDRGs disclosed herein.

The aforementioned polypeptides may be obtained by any of a variety of methods. Smaller peptides (less than 50 amino acids long) are conveniently synthesized by standard chemical techniques and can be chemically or enzymatically ligated to form larger polypeptides. Polypeptides can be purified from biological sources by methods well known in the art, for example, as described in Protein Purification, Principles and Practice, Second Edition Scopes, Springer Verlag, N.Y. (1987) Polypeptides are optionally but preferably produced in their naturally occurring, truncated, or fusion protein forms by recombinant DNA technology using techniques well known in the art. These methods include, for example, in vitro recombinant DNA techniques, synthetic techniques and in vivo genetic recombination. See, for example, the techniques described in Sambrook et al. (2001) Molecular Cloning, A Laboratory Manual, Third Edition, Cold Spring Harbor Press, N.Y.; and Ausubel et al., eds. (1997) Current Protocols in Molecular Biology, Green Publishing Associates, Inc., and John Wiley & Sons, Inc., N.Y. (supplemented through 2002). RNA encoding the proteins may also be chemically synthesized. See, for example, the techniques described in Oligonucleotide Synthesis, (1984) Gait ed., IRL Press, Oxford, which is incorporated by reference herein in its entirety.

The nucleic acid molecules described herein may be expressed in a suitable host cell or an organism to produce proteins. Expression may be achieved by placing a nucleotide sequence encoding these proteins into an appropriate expression vector and introducing the expression vector into a suitable host cell, culturing the transformed host cell under conditions suitable for expression of the proteins described or variants thereof, or a polypeptide that comprises one or more domains of such proteins. The recombinant proteins from the host cell may be purified to obtain purified and, preferably, active protein. Alternatively, the expressed protein may be allowed to function in the intact host cell or host organism.

Appropriate expression vectors are known in the art, and may be purchased or applied for use according to the manufacturer's instructions to incorporate suitable genetic modifications. For example, pET-14b, pcDNA1Amp, and pVL1392 are available from Novagen and Invitrogen, and are suitable vectors for expression in E. coli, mammalian cells and insect cells, respectively. These vectors are illustrative of those that are known in the art, and many other vectors can be used for the same purposes. Suitable host cells can be any cell capable of growth in a suitable media and allowing purification of the expressed protein. Examples of suitable host cells include bacterial cells, such as E. coli, Streptococci, Staphylococci, Streptomyces and Bacillus subtilis cells; fungal cells such as Saccharomyces and Aspergillus cells; insect cells such as Drosophila S2 and Spodoptera Sf9 cells, mammalian cells such as CHO, COS, HeLa, 293 cells; and plant cells.

Culturing and growth of the transformed host cells can occur under conditions that are known in the art. The conditions will generally depend upon the host cell and the type of vector used. Suitable culturing conditions may be used such as temperature and chemicals and will depend on the type of promoter utilized.

Purification of the proteins or domains of such proteins, if desired, may be accomplished using known techniques without performing undue experimentation. Generally, the transformed cells expressing one of these proteins are broken, crude purification occurs to remove debris and some contaminating proteins, followed by chromatography to further purify the protein to the desired level of purity. Host cells may be broken by known techniques such as homogenization, sonication, detergent lysis and freeze-thaw techniques. Crude purification can occur using ammonium sulfate precipitation, centrifugation or other known techniques. Suitable chromatography includes anion exchange, cation exchange, high performance liquid chromatography (HPLC), gel filtration, affinity chromatography, hydrophobic interaction chromatography, etc. Well known techniques for refolding proteins can be used to obtain the active conformation of the protein when the protein is denatured during intracellular synthesis, isolation or purification.

In general, RSDRG proteins or domains, or antibodies to such proteins can be purified, either partially (e.g., achieving a 5×, 10×, 100×, 500×, or 1000× or greater purification), or even substantially to homogeneity (e.g., where the protein is the main component of a solution, typically excluding the solvent (e.g., water or DMSO) and buffer components (e.g., salts and stabilizers) that the protein is suspended in, e.g., if the protein is in a liquid phase), according to standard procedures known to and used by those of skill in the art. Accordingly, the polypeptides can be recovered and purified by any of a number of methods well known in the art, including, e.g., ammonium sulfate or ethanol precipitation, acid or base extraction, column chromatography, affinity column chromatography, anion or cation exchange chromatography, phosphocellulose chromatography, hydrophobic interaction chromatography, hydroxylapatite chromatography, lectin chromatography, gel electrophoresis and the like. Protein refolding steps can be used, as desired, in making correctly folded mature proteins. High performance liquid chromatography (HPLC), affinity chromatography or other suitable methods can be employed in final purification steps where high purity is desired. In one embodiment, antibodies made against the proteins described herein are used as purification reagents, e.g., for affinity-based purification of proteins comprising one or more RSDRG protein domains or antibodies thereto. Once purified, partially or to homogeneity, as desired, the polypeptides are optionally used e.g., as assay components, therapeutic reagents or as immunogens for antibody production.

In addition to other references noted herein, a variety of purification methods are well known in the art, including, for example, those set forth in R. Scopes, Protein Purification, Springer-Verlag, N.Y. (1982); Deutscher, Methods in Enzymology Vol. 182: Guide to Protein Purification, Academic Press, Inc. N.Y. (1990); Sandana, Bioseparation of Proteins, Academic Press, Inc. (1997); Bollag et al., Protein Methods, 2nd Edition Wiley-Liss, NY; Walker (1996) The Protein Protocols Handbook Humana Press, NJ; Harris and Angal Protein Purification Applications: A Practical Approach IRL Press at Oxford, Oxford, England (1990); Scopes, Protein Purification. Principles and Practice 3rd Edition Springer Verlag, NY (1993); Janson and Ryden, Protein Purification: Principles, High Resolution Methods and Applications, Second Edition Wiley-VCH, NY (1998); and Walker, Protein Protocols on CD-ROM Humana Press, NJ (1998); and the references cited therein.

After synthesis, expression and/or purification, proteins may possess a conformation different from the desired conformations of the relevant polypeptides. For example, polypeptides produced by prokaryotic systems often are optimized by exposure to chaotropic agents to achieve proper folding. During purification from, e.g., lysates derived from E. coli, the expressed protein is optionally denatured and then renatured. This is accomplished, e.g., by solubilizing the proteins in a chaotropic agent such as guanidine HCl. In general, it is occasionally desirable to denature and reduce expressed polypeptides and then to cause the polypeptides to re-fold into the preferred conformation. For example, guanidine, urea, DTT, DTE, and/or a chaperonin can be added to a translation product of interest. Methods of reducing, denaturing and renaturing proteins are well known to those of skill in the art. Debinski, et al., for example, describe the denaturation and reduction of inclusion body proteins in guanidine-DTE. The proteins can be refolded in a redox buffer containing, e.g., oxidized glutathione and L-arginine. Refolding reagents can be flowed or otherwise moved into contact with the one or more polypeptide or other expression product, or vice-versa.

In another aspect, antibodies to the RSDRG proteins or fragments thereof may be generated using methods that are well known in the art. The antibodies may be utilized for detecting and/or purifying the RSDRG proteins, optionally discriminating the proteins from various homologues. As used herein, the term “antibody” includes, but is not limited to, polyclonal antibodies, monoclonal antibodies, humanized or chimeric antibodies and biologically functional antibody fragments, which are those fragments sufficient for binding of the antibody fragment to the protein.

General protocols that may be adapted for detecting and measuring the expression of the described RSDRG proteins using the above mentioned antibodies are known. Such methods include, but are not limited to, dot blotting, western blotting, competitive and noncompetitive protein binding assays, enzyme-linked immunosorbant assays (ELISA), immunohistochemistry, fluorescence-activated cell sorting (FACS), and other protocols that are commonly used and widely described in scientific and patent literature.

Sequence of the RSDRG genes may also be used in genetic mapping of plants or in plant breeding. Polynucleotides derived from the RSDRG gene sequences may be used in in situ hybridization to determine the chromosomal locus of the RSDRG genes on the chromosomes. These polynucleotides may also be used to detect segregation of different alleles at certain RSDRG loci.

Sequence information of the RSDRG genes may also be used to design oligonucleotides for detecting RSDRG mRNA levels in the cells or in plant tissues. For example, the oligonucleotides can be used in a Northern blot analysis to quantify the levels of RSDRG mRNA. Moreover, full-length or fragment of the RSDRG genes may be used in preparing microarrays (or gene chips). Full-length or fragment of the RSDRG genes may also be used in microarray experiments to study expression profile of the RSDRG genes. High-throughput screening can be conducted to measure expression levels of the RSDRG genes in different cells or tissues. Various compounds or other external factors may be screened for their effects expression of the RSDRG gene expression.

Sequences of the RSDRG genes and proteins may also provide a tool for identification of other proteins that may be involved in plant drought response. For example, chimeric RSDRG proteins can be used as a “bait” to identify other proteins that interact with RSDRG proteins in a yeast two-hybrid screening. Recombinant RSDRG proteins can also be used in pull-down experiment to identify their interacting proteins. These other proteins may be cofactors that enhance the function of the RSDRG proteins, or they may be RSDRG proteins themselves which have not been identified in the experiments disclosed herein.

The RSDRG polypeptides may possess structural features which can be recognized, for example, by using immunological assays. The generation of antisera which specifically bind the RSDRG polypeptides, as well as the polypeptides which are bound by such antisera, are a feature of the disclosed embodiments.

In order to produce antisera for use in an immunoassay, one or more of the immunogenic RSDRG polypeptides or fragments thereof are produced and purified as described herein. For example, recombinant protein may be produced in a host cell such as a bacterial or an insect cell. The resultant proteins can be used to immunize a host organism in combination with a standard adjuvant, such as Freund's adjuvant. Commonly used host organisms include rabbits, mice, rats, donkeys, chickens, goats, horses, etc. An inbred strain of mice may also be used to obtain more reproducible results due to the virtual genetic identity of the mice. The mice are immunized with the immunogenic RSDRG polypeptides in combination with a standard adjuvant, such as Freund's adjuvant, and a standard mouse immunization protocol. See, for example, Harlow and Lane, Antibodies, A Laboratory Manual, Cold Spring Harbor Publications, New York (1988), which provides comprehensive descriptions of antibody generation, immunoassay formats and conditions that can be used to determine specific immunoreactivity. Alternatively, one or more synthetic or recombinant RSDRG polypeptides or fragments thereof derived from the sequences disclosed herein is conjugated to a carrier protein and used as an immunogen.

Antisera that specifically bind the RSDRG proteins may be used in a range of applications, including but not limited to immunofluorescence staining of cells for the expression level and localization of the RSDRG proteins, cytological staining for the expression of RSDRG proteins in tissues, as well as in Western blot analysis.

Another aspect of the disclosure includes screening for potential or candidate modulators of RSDRG protein activity. For example, potential modulators may include small molecules, organic molecules, inorganic molecules, proteins, hormones, transcription factors, or the like, which can be contacted to a cell or certain tissues that express the RSDRG proteins to assess the effects, if any, of the candidate modulator upon RSDRG protein activity.

Alternatively, candidate modulators may be screened to modulate expression of RSDRG proteins. For example, potential modulators may include small molecules, organic molecules, inorganic molecules, proteins, hormones, transcription factors, or the like, which can be contacted to a cell or certain tissues that express the RSDRG proteins, to assess the effects, if any, of the candidate modulator upon RSDRG protein expression. Expression of a RSDRG gene described herein may be detected, for example, via Northern blot analysis or quantitative (optionally real time) RT-PCR, before and after application of potential expression modulators. Alternatively, promoter regions of the various RSDRG genes may be coupled to reporter constructs including, without limitation, CAT, beta-galactosidase, luciferase or any other available reporter, and may similarly be tested for expression activity modulation by the candidate modulator. Promoter regions of the various genes are generally sequences in the proximity upstream of the start site of transcription, typically within 1 Kb or less of the start site, such as within 500 bp, 250 bp or 100 bp of the start site. In certain cases, a promoter region may be located between 1 and 5 Kb from the start site.

In either case, whether the assay is to detect modulated activity or expression, a plurality of assays may be performed in a high-throughput fashion, for example, using automated fluid handling and/or detection systems in serial or parallel fashion. Similarly, candidate modulators can be tested by contacting a potential modulator to an appropriate cell using any of the activity detection methods herein, regardless of whether the activity that is detected is the result of activity modulation, expression modulation or both.

A method of modifying a plant may include introducing into a host plant one or more RSDRG genes described above. The RSDRG genes may be placed in an expression construct, which may be designed such that the RSDRG protein(s) are expressed constitutively, or inducibly. The construct may also be designed such that the RSDRG protein(s) are expressed in certain tissue(s), but not in other tissue(s). The RSDRG protein(s) may enhance the ability of the host plant in drought tolerance, such as by reducing water loss or by other mechanisms that help a plant cope with water deficit growth conditions. The host plant may include any plants whose growth and/or yield may be enhanced by a modified drought response. Methods for generating such transgenic plants is well known in the field. See e.g., Leandro Peña (Editor), Transgenic Plants: Methods and Protocols (Methods in Molecular Biology), Humana Press, 2004.

The use of gene inhibition technologies such as antisense RNA or co-suppression or double stranded RNA interference is also within the scope of the present disclosure. In these approaches, the isolated gene sequence is operably linked to a suitable regulatory element. In one embodiment of the disclosure, the construct contains a DNA expression cassette that contains, in addition to the DNA sequences required for transformation and selection in said cells, a DNA sequence that encodes a RSDRG proteins or a RSDRG modulator protein, with at least a portion of said DNA sequence in an antisense orientation relative to the normal presentation to the transcriptional regulatory region, operably linked to a suitable transcriptional regulatory region such that said recombinant DNA construct expresses an antisense RNA or portion thereof of an antisense RNA in the resultant transgenic plant.

It is apparent to one of skill in the art that the polynucleotide encoding the RSDRG proteins or a RSDRG modulator proteins can be in the antisense (for inhibition by antisense RNA) or sense (for inhibition by co-suppression) orientation, relative to the transcriptional regulatory region. Alternatively a combination of sense and antisense RNA expression can be utilized to induce double stranded RNA interference. See, e.g., Chuang and Meyerowitz, PNAS 97: 4985-4990, 2000; see also Smith et al., Nature 407: 319-320, 2000.

These methods for generation of transgenic plants generally entail the use of transformation techniques to introduce the gene or construct encoding the RSDRG proteins or a RSDRG modulator proteins, or a part or a homolog thereof, into plant cells. Transformation of a plant cell can be accomplished by a variety of different methodology. Methods that have general utility include, for example, Agrobacterium based systems, using either binary and/or cointegrate plasmids of both A. tumifaciens and A. rhyzogenies, (See e.g., U.S. Pat. No. 4,940,838, U.S. Pat. No. 5,464,763), the biolistic approach (See e.g., U.S. Pat. No. 4,945,050, U.S. Pat. No. 5,015,580, U.S. Pat. No. 5,149,655), microinjection, (See e.g., U.S. Pat. No. 4,743,548), direct DNA uptake by protoplasts, (See e.g., U.S. Pat. No. 5,231,019, U.S. Pat. No. 5,453,367) or needle-like whiskers (See e.g., U.S. Pat. No. 5,302,523). Any method for the introduction of foreign DNA into a plant cell and for expression therein may be used within the context of the present disclosure.

Plants that are capable of being transformed encompass a wide range of species, including but not limited to soybean, corn, potato, rice, wheat and many other crops, fruit plants, vegetables and tobacco. See generally, Vain, P., Thirty years of plant transformation technology development, Plant Biotechnol J. 2007 March; 5(2):221-9. Any plants that are capable of taking in foreign DNA and transcribing the DNA into RNA and/or further translating the RNA into a protein may be a suitable host.

The modulators described above that may alter the expression levels or the activity of the RSDRG proteins (collectively called RSDRG modulators) may also be introduced into a host plant in the same or similar manner as described above. In one embodiment, the RSDRG modulators are primarily transcription factors that regulate the transcription of the RSDRGs.

The RSDRG proteins or the RSDRG modulators may be used to modify a target plant by causing them to be assimilated by the plant. Alternatively, the RSDRG proteins or the RSDRG modulators may be applied to a target plant by causing them to be in contact with the plant, or with a specific organ or tissue of the plant. In one embodiment, organic or inorganic molecules that can function as RSDRG modulators may be caused to be in contact with a plant such that these chemicals may enhance the drought response of the target plant.

In addition to the RSDRG modulators, RSDRG polypeptides or RSDRG nucleic acids, a composition containing other ingredients may be introduced, administered or delivered to the plant to be modified. In one aspect, a composition containing an agriculturally acceptable ingredient may be used in conjunction with the RSDRG modulators to be administered or delivered to the plant.

Bioinformatic systems are widely used in the art, and can be utilized to identify homology or similarity between different character strings, or can be used to perform other desirable functions such as to control output files, provide the basis for making presentations of information including the sequences and the like. Examples include BLAST, discussed supra. For example, commercially available databases, computers, computer readable media and systems may contain character strings corresponding to the sequence information herein for the RSDRG polypeptides and nucleic acids described herein. These sequences may include specifically the RSDRG sequences listed herein and the various silent substitutions and conservative substitutions thereof.

The bioinformatic systems contain a wide variety of information that includes, for example, a complete sequence listings for the entire genome of an individual organism representing a species. Thus, for example, using the RSDRG sequences as a basis for comparison, the bioinformatic systems may be used to compare different types of homology and similarity of various stringency and length on the basis of reported data. These comparisons are useful to identify homologs or orthologs where, for example, the basic RSDRG gene ortholog is shown to be conserved across different organisms. Thus, the bioinformatic systems may be used to detect or recognize the homologs or orthologs, and to predict the function of recognized homologs or orthologs. By way of example, many homology determination methods have been designed for comparative analysis of sequences of biopolymers including nucleic acids, proteins, etc. With an understanding of hydrogen bonding between the principal bases in natural polynucleotides, models that simulate annealing of complementary homologous polynucleotide strings can also be used as a foundation of sequence alignment or other operations typically performed on the character strings corresponding to the sequences herein. One example of a software package for calculating sequence similarity is BLAST, which can be adapted to the present invention by inputting character strings corresponding to the sequences herein.

The software can also include output elements for controlling nucleic acid synthesis (e.g., based upon a sequence or an alignment of a sequences herein) or other operations which occur downstream from an alignment or other operation performed using a character string corresponding to a sequence herein.

In an additional aspect, kits may embody any of the methods, compositions, systems or apparatus described above. Kits may optionally comprise one or more of the following: (1) a composition, system, or system component as described herein; (2) instructions for practicing the methods described herein, and/or for using the compositions or operating the system or system components herein; (3) a container for holding components or compositions, and, (4) packaging materials.

### EXAMPLES

The following nonlimiting examples report general procedures, reagents and characterization methods that teach by way of example, and should not be construed in a narrowing manner that limits the disclosure to what is specifically disclosed. Those skilled in the art will understand that numerous modifications may be made and still the result will fall within the spirit and scope of the present invention.

### Example 1

### Differential Gene Expression of Drought Response Genes in Soybean Roots

The overall goal of these experiments is to identify potential gene(s) that may confer upon a plant the capability to grow and reproduce under water deficit conditions. The first step towards accomplishing this goal is to characterize the transcription profiles in regions of growth maintenance and inhibition in soybean roots under water deficit conditions in comparison to well-watered conditions. Another approach taken in this disclosure is to compare the transcription profiles in the root regions of soybean with those from other plants, such as maize. One specific objective is to characterize the transcriptome of apical and basal regions of soybean root growth zone under water deficit conditions for a short (5 h) and long (48 h) period. Soybean Genechips manufactured by Affymetrix were used.

Briefly, young Magellan soybean plants were subject to water deficit stress treatment for 5 h or 48 h. Root regions 1 and 3 from well watered samples and root regions 1 and 2 from water stressed (or deficit) samples were collected in triplicate. Total RNA isolation and microarray hybridizations were conducted using standard protocols such as the protocols described in U.S. patent application Ser. No. 12/138,392 (US2009/0210968). 60K soybean Affymetrix GeneChip (total 30) and the pair wise comparison experimental plan were used for the microarray experiments.

More specifically, a soybean seedling system was used for these experiments. Soybean (Glycine max L.) line Magellan seeds were sterilized with 20% bleach for 2 minutes and rinsed with running tap water for about 20 minutes. Seeds were then placed on germination paper saturated with 10 mM CaCl₂ and 10 mM Ca(NO₃)₂ solutions and germinated in the dark at 29° C. and nearly 100% relative humidity. A mixture of vermiculite and Turface at a 1:1 volumetric ratio was used as the culture medium. Plastic tubes covered with fiberglass mesh at the bottom and plastic boxes were used as growth containers and were filled with either well-watered (pre-soaked with 10 mM CaCl₂ and 10 mM Ca(NO₃)₂ solution) or water-stressed medium. The water-stressed medium was pre-mixed with 10 mM CaCl₂ and 10 mM Ca(NO₃)₂ solutions to a water potential of about −1.6 MPa as measured with isopiestic thermocouple psychrometers (Boyer and Knipling, 1965). The plastic tubes and boxes containing culture medium were then placed in the humid room, where the parameters were set for seedling growth (temperature: 29° C. and 100% RH, no light), for one day before transplanting.

Uniform seedlings with a root length of 11-25 mm were transplanted to boxes and tubes containing either well-watered or water-stressed medium. At 5 h, 12 h, 24 h, and 48 hours after transplanting, three biological replicates of soybean seedlings were harvested. Samples from 5 h and 48 h timpoints (earlier and later stages of water deficit) after transplanting were used for the microarray analysis. Samples from 5 h, 12 h, 24 h, and 48 hours after transplanting were used to conduct qRTPCR analysis of the transcription factors and other selected genes from the microarray results. The 5 h, 12 h, and 24 h time points were chosen to permit the identification of early-responsive transcription factors after the initiation of water-stress treatment and before root growth reached the steady state (Sharp et al. 2004). The 48 h time point was selected to identify transcription factors that might be responsible for the changes associated with the steady-state patterns of the relative elongation rate of soybean seedling roots in response to water-stress conditions.

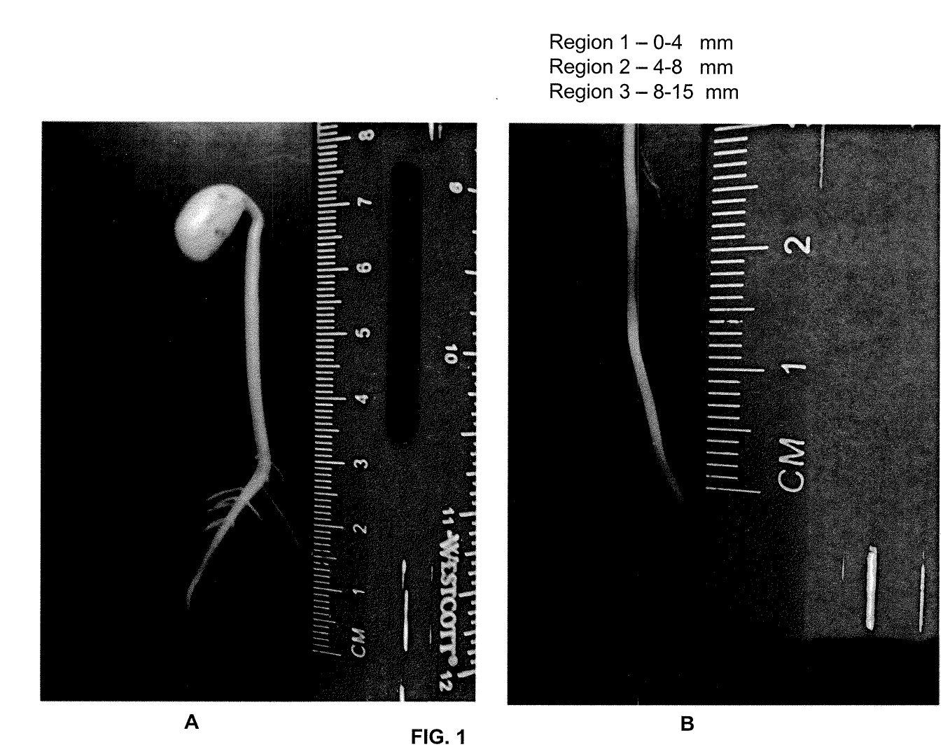

The soybean seedlings were grown to about 6-10 cm tall. The primary roots of these soybeans seedlings were dissected into 3 regions based on previously characterized expansion profiles: Region 1(R1) is 0-4 mm from the root tip (including the root cap), Region 2 (R2) is 4-8 mm from the root tip, and Region 3 (R3) is 8-15 mm from the root tip. See FIG. 1. Regions 1-2 and regions 1-3 were collected in water-stressed roots and well-watered roots, respectively. These root regions were immediately frozen in liquid nitrogen and then stored at −80° C. for RNA isolation and microarray analysis. Comparison of gene expression between region 2 of water-stressed roots and region 3 of well-watered roots may help distinguish the responses induced by water stress from responses resulting from changes in normal growth deceleration.

The soybean primary root adapts to low water potential (e.g., Ψ_{w}, −1.6 MPa) by maintaining longitudinal expansion in the apical 4 mm (region 1), whereas in the adjacent 4 mm (region 2) longitudinal expansion reaches a maximum in well-watered roots but is progressively inhibited at low Ψ_{w} (Yamaguchi et al. 2009). To identify mechanisms that determine these responses to low Ψ_{w}, and to elucidate the regulatory networks involved, the gene expression patterns in these regions of water-stressed and well-watered roots were profiled. Also, the gene expression between region 2 of water-stressed roots and the growth deceleration zone in well-watered roots (region 3) were compared to help distinguish stress-responsive genes in region 2 from those involved in cell maturation. The results reveal that stress-responsive transcripts are largely root region specific. The differentially expressed transcripts (also termed “DET”) were then compared to the available soybean genome sequence information, and the major metabolic and transcriptional regulatory pathways involved in the response to water deficit conditions.

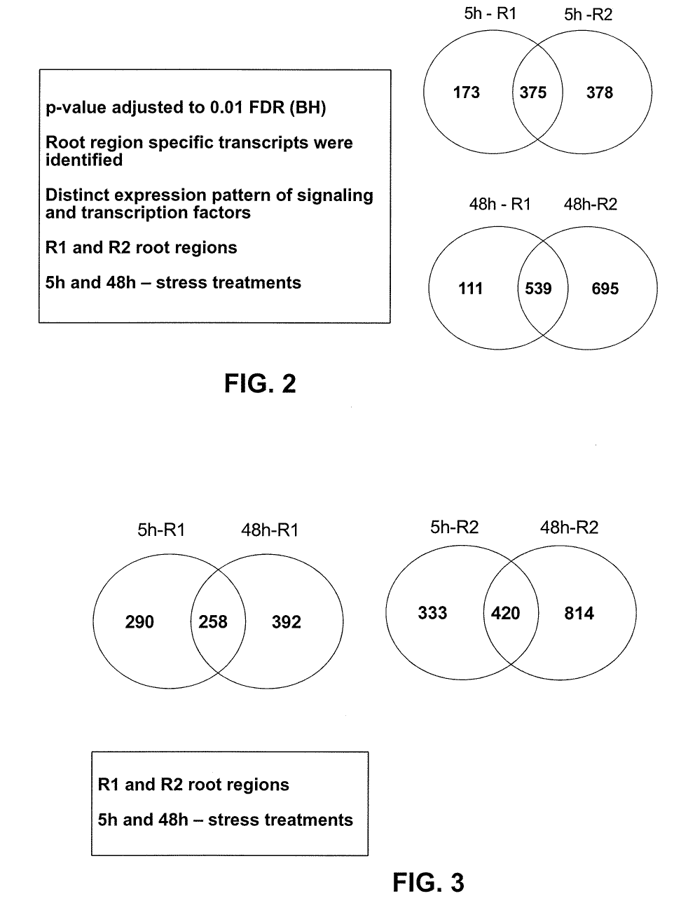

FIG. 2 shows the numbers of genes that showed differential expression at the same time point between different root regions. As shown in the upper panel of FIG. 2, 548 (173+375) genes show differential expression in the R1 region after 5 hour under drought conditions (Table 1). By contrast, 753 (378+375) genes show differential expression in the R2 region after 5 hour under drought conditions (Table 2). Among these genes, 375 genes are differentially expressed after 5 hour under water deficit conditions in both the R1 and R2 regions (Table 3), while 173 genes show R1 specific differential expression and 378 genes show R2 specific differential expression at the 5 hour time point.

As shown in the lower panel of FIG. 2, 650 (111+539) genes show differential expression in the R1 region after 48 hour under drought conditions (Table 4). By contrast, 1234 (695+539) genes show differential expression in the R2 region after 48 hour under drought conditions (Table 5). Among these genes, 539 genes are differentially expressed after 48 hour under water deficit conditions in both the R1 and R2 regions (Table 6), while 111 genes show R1 specific differential expression and 695 genes show R2 specific differential expression at the 48 hour time point.

The number of genes that are differentially expressed within the same root region are compared between the two time points, namely, 5 hr and 48 hr after the plants have been subjected to water deficit conditions (FIG. 3). Out of the 548 genes found to be differentially expressed in the R1 region after 5 hour under water deficit conditions (Table 7), 290 genes are differentially expressed in the R1 region only after 5 hours, but not after 48 hours, under drought conditions (Table 8). Out of the 650 genes found to be differentially expressed after 48 hour under water deficit conditions (Table 9), 392 genes are differentially expressed in the R1 region only after 48 hours, but not after 5 hours, under drought conditions (Table 10). Similarly, out of the 753 genes found to be differentially expressed in the R2 region after 5 hour under water deficit conditions (Table 11), 333 genes are differentially expressed in the R1 region only after 5 hours, but not after 48 hours, under drought conditions (Table 12). Out of the 1234 genes found to be differentially expressed in the R2 region after 48 hour under water deficit conditions (Table 13), 814 genes are differentially expressed in the R2 region only after 48 hours, but not after 5 hours, under drought conditions (Table 14).

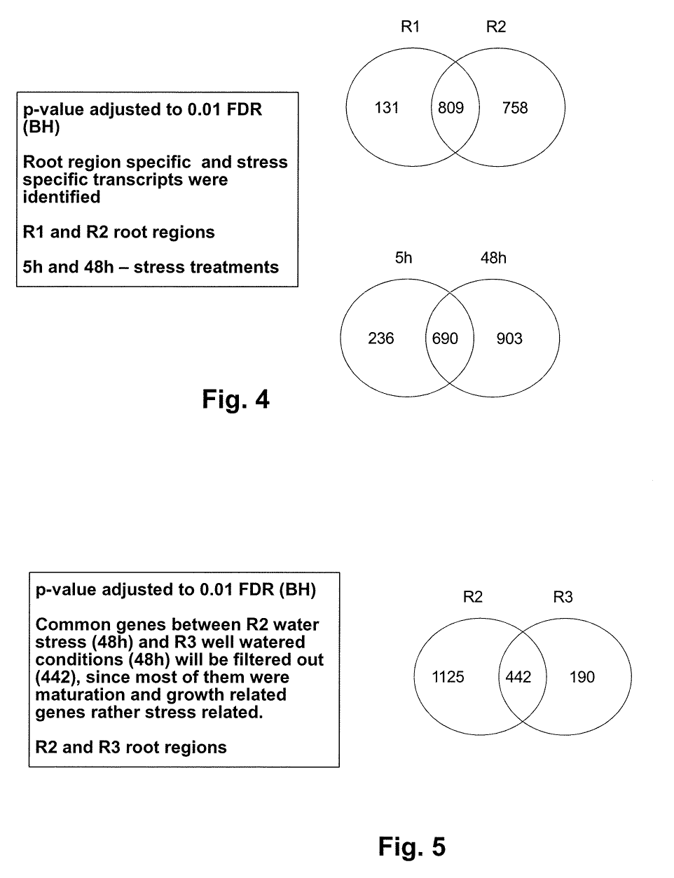

The upper panel of FIG. 4 shows genes that are only differentially expressed in R1, R2 or both R1 and R2 regions of the root up to 48 hours after the plants are exposed to drought conditions, which include the 5 h and 48 h time points. Out of a total of 940 (290+258+392 as shown in FIG. 3) genes that are differentially expressed at 5 hr and 48 hour after being exposed to drought conditions, 131 genes are only differentially expressed in the R1 region (Table 15). Out of a total of 1567 (333+420+814 as shown in FIG. 3) genes that are differentially expressed at 5 hr and 48 hour after being exposed to drought conditions, 758 genes show differential expression under drought conditions only in R2 region of the root (Table 16). Interestingly, 809 genes show differential expression in both R1 and R2 regions after 5 hr or 48 hour exposure to drought conditions.

As shown in the lower panel of FIG. 4, out of the total of 926 genes that show differential expression in both R1 and R2 regions 5 hours after being subjected to drought conditions, 236 genes show differential expression after 5 hour, but not after 48 hours exposure to drought conditions. Differential expression was observed in 903 genes only after 48 hours, but not at 5 hours after exposure to drought conditions. 690 genes show differential expression both at 5 hours and 48 hours after exposure to drought conditions.

As shown in FIG. 5, 1125 genes were found to be differentially expressed under drought condition only in the R2 region but not in the R3 region. Conversely, 190 genes are differentially expressed under drought condition only in the R3 region but not in the R2 region. The R2 and R3 regions also share 442 genes that are commonly expressed in both regions under drought conditions. Most of these 442 genes likely represent genes that are implicated in maturation and/or growth of the roots, but are not specifically induced by the water deficit conditions.

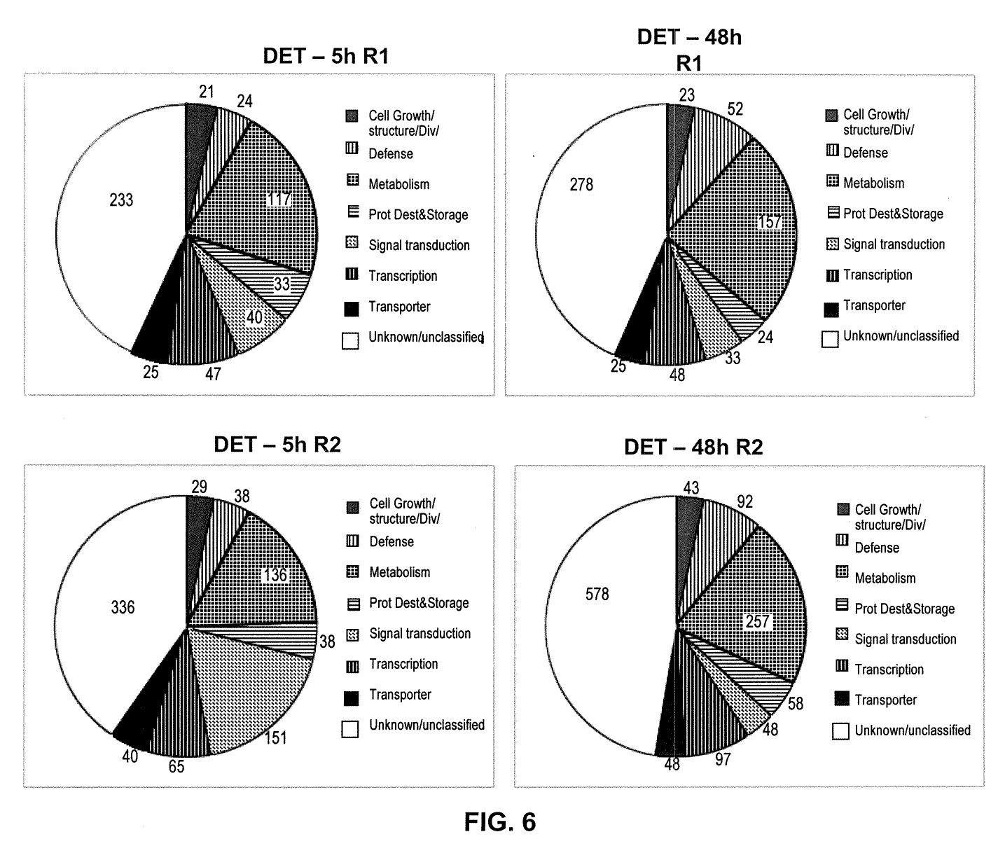

FIG. 6 presents pie charts illustrating the different functional categories of genes that show differential expression in the R1 (8A and 8B) or R2 (8C and 8D) regions after having been subjected to water deficit conditions for 5 hour (8A and 8C) or 48 hours (8B and 8D). DET stands for “Differentially Expressed Transcripts.”

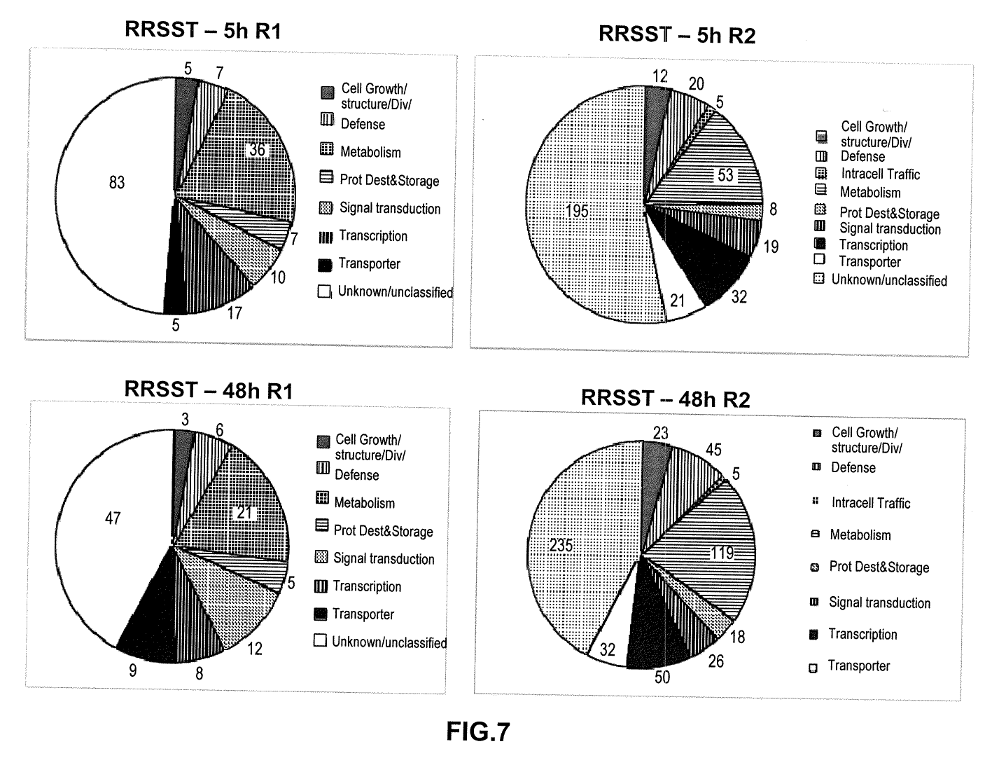

FIG. 7 shows the different categories of the “root region and stress specific transcripts” (RRSST) by their known or predicted function.

### Example 2

### Comparison of the Gene Expression Patterns Under Drought Conditions in the Root Regions of Soybean and Maize

To gain insight into whether the gene expression pattern in response to drought differs among different species, the root region specific and stress specific gene expression patterns of monocot (Maize) and dicot (Soybean) were compared. These analyses confirmed that a species-specific gene expression pattern exists under water deficit conditions in root tissues.

Microarray studies were conducted in maize using the same seedling root experimental conditions as used in the soybean system described in Example 1. Based on the maize array results (root region specific and stress specific genes) and the results from other experiments and also from the reported gene information in the literature, a panel/subset of 273 genes were selected for further testing. In order to further characterize the gene expression under various time points of water deficit conditions in maize roots, high throughput qRT-PCR was utilized for more precise and sensitive data points. These 273 genes (listed in Table 17) were found to be differentially expressed in various root regions of maize at different stress time points (0, 3, 6, 12, 24, 48 hours of stress). The actual sequences of 224 genes out of these 273 obtained from the maize genome database are disclosed herein as SEQ ID. Nos. 1-224. The remaining 49 genes all have GenBank accession numbers whose sequences can be obtained for free from the GenBank database maintained by NCBI.

Because the root system of soybean and maize are similar, it is likely that soybean homologs of these genes also play some roles in stress avoidance mechanism by maintaining root growth under water deficit conditions. Soybean homologs of these 273 maize genes are identified by Blast searching and the sequences of some of these soybean homologs are disclosed herein as SEQ ID. Nos. 225-432.

A number of genes were found to show differential expression in root region R1 5 hours after being subjected to drought conditions in both soybean and maize. Examples of such genes include root specific protein RCc3, ionositol oxygenase, Lipoxygenase, among others. A number of such genes are listed in Table 18 showing sequence comparison between the drought response genes from soybean and their counterparts from maize.

At least 30 common genes show differential expression in root region R25 hours after being subjected to drought conditions in both soybean and maize. Examples of such genes include bZip, DREB, ACC oxi, CDPK, etc. Table 19 lists some of these genes from both soybean and maize and the sequence similarity between the soybean genes and their respective maize counterparts.

At least 18 genes show differential expression in root region R1 after 48 hours under drought conditions in both soybean and maize. Examples of such genes include put. NAC, CCAAT, put. Myb, GST, etc. Table 20 lists some of these genes from both soybean and maize and the sequence similarity between the soybean genes and their respective maize counterparts.

At least 29 genes show differential expression in root region R2 after 48 hour under drought conditions in both soybean and maize. Examples of such genes include NAC, a-Galact, CCAAT, SAPK3, HSP20, etc. Table 21 lists some of these genes from both soybean and maize and the sequence similarity between the soybean genes and their respective maize counterparts.

As described above, microarray experiments were performed in maize and soybean seedling root system with substantially the same water deficit conditions and similar root segment size. Soybean genes differentially expressed under water deficit conditions were compared with the maize microarray results. Those soybean genes common between soybean and maize whose expression was found to be altered under water deficit conditions were identified. The sequences of these soybean genes are disclosed herein as SEQ ID. Nos. 433-480. Because these genes have homologs in both soybean and maize whose expression levels are altered in response to drought conditions, these genes are likely to encode proteins that play a role in drought response in a plant.

### Example 3

### Confirmation of Root Drought Response Genes and Creation of Transgenic Plants

The genes shown to be up-regulated or down-regulated were confirmed by comparing the expression levels of specific genes in the root regions under drought or normal conditions by using Northern blot or quantitative RT-PCR. Genes that have been confirmed were cloned and placed in different expression constructs so that they could be introduced into different host plant.

The genes found to be upregulated or downregulated in the root regions of soybean and/or maize may be altered in a host plant such as soybean or maize in order to generate genetically altered plants that are more resistant to water deficit conditions. Alternatively, one or more of these genes may be introduced into a host plant and forced to be expressed at an elevated level or at a specific location in order to create a genetically altered plant that is more tolerant to water deficit conditions.

Table 22 shows the names of the genes that have been confirmed and selected to be introduced into a host plant, such as Arabidopsis, soybean and maize in order to obtain transgenic plants that are more tolerant to drought conditions than the non-transgenic host plant. The sequences of most of the genes listed in Table 22 are also disclosed as SEQ ID Nos. 481-510. Other sequences can be obtained from the GenBank database or various soybean, maize and arabidopsis databases. The promoters used for most of the constructs are also disclosed in Table 22.

While the foregoing instrumentalities have been described in some detail for purposes of clarity and understanding, it will be clear to one skilled in the art from a reading of this disclosure that various changes in form and detail can be made without departing from the true scope of the invention. For example, all the techniques and apparatus described above may be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

### REFERENCES

In addition to those references that are cited in full in the text, additional information for those abbreviated citations is listed below. The content of all patents, patent applications or other publications cited in this disclosure are incorporated by reference into this disclosure.
Boyer, J S, 1983, Environmental stress and crop yields. In C. D. Raper and P. J. Kramer (ed) Crop reactions to water and temperature stresses In humid, temperature climates. Westview press, Boulder, Colo. pp 3-7.
Muchow R C, Sinclair T R. 1988. Water and nitrogen limitations In soybean grain production. II. Field and model analyses. Field Crop Res. 15:143-158.
Specht J E, Hume D J, Kumind S V. 1999. Soybean yield potential-A genetic physiological perspective. Crop Science 39:1560-1570.
Neumann P M: Coping mechanisms for crop plants in drought-prone environments. Ann Bot 2008, 101:901-907.
Wang W, Vinocur B, Altman A: Plant responses to drought, salinity and extreme temperatures: towards genetic engineering for stress tolerance. Planta 2003, 218:1-14.
Vinocur, B, Altman A: Recent advances in engineering plant tolerance to abiotic stress: achievements and limitations. Curr Opin Biotech 2005, 16:123-32.
Chaves M M, Oliveire M M: Mechanisms underlying plant resilience to water deficits: prospects for water-saving agriculture. J Exp Bot 2004, 55; 2365-2384.
Shinozaki K, Yamaguchi-Shinozaki K, Seki M: Regulatory network of gene expression in the drought and cold stress responses. Curr Opin Plant Biol 2003, 6:410-417.
Schena M, Shalon D, Davis R W, Brown P O (1995) Quantitative monitoring of gene expression patterns with a complementary DNA microarray. Science 270: 467-470
Shalon D, Smith S, Brown P (1990) A DNA microarray system for analyzing complex DNA samples using two-color fluorescent probe hybridization. Genome Res. 8: 639-645.
Bray E A: Genes commonly regulated by water-deficit stress in Arabidopsis thaliana. J Exp Bot 2004, 55:2331-2341.
Denby K, Gehring C: Engineering drought and salinity tolerance in plants: lessons from genome-wide expression profiling In Arabidopsis. Trends in Plant Sci 2005, 23547-552.
Shinozaki K, Yamaguchi-Shinozaki K: Molecular responses to drought and cold stress. Curr Opin Biotech 1996, 7:181-167
Shinozaki. K. and Yamaguchi-Shinozaki, K: Molecular responses to dehydration and low temperature; differences and cross-talk between two stress signaling pathways. Curr Opin Plant Biol 2000, 3:217-223.
Seki M, Narusaka M, Abe H, Kasuga M, Yamaguchi-Shinozaki K, Carninci P, Hayashizaki Y, Shinozaki K: Monitoring the expression pattern of 1300 Arabidopsis genes under drought and cold stresses by using a full-length cDNA microarray. Plant Cell 2001, 13:61-72.
Fowler S, Thomashow M F: Arabidopsis transcriptome profiling indicates that multiple regulatory pathways are activated during cold acclimation In addition to the CBF cold response pathway, Plant Cell 2002, 14:1875-1690.
Maruyama K, Sakuma Y, Kasuga M, Ito Y, Seki M, Goda H, Shimada Y, Yoshida S, Shinozaki K, Yamaguchi-Shinozaki K: Identification of cold-inducible downstream genes of the Arabidopsis DREB1A/CBF3 transcriptional factor using two microarray systems. Plant J 2004, 38:982-993.
Yamaguchi M, Valliyodan B, Zhang J, LeNoble M E, Yu O, Nguyen H T, Sharp R E Proteomic analysis of the differential responses of elongation rate to water stress within the soybean primary root growth zone. Plant, Cell & Environment 2009. [Epub ahead of print]

Lengthy table referenced here
US20110119792A1-20110519-T00001
Please refer to the end of the specification for access instructions.

Lengthy table referenced here
US20110119792A1-20110519-T00002
Please refer to the end of the specification for access instructions.

Lengthy table referenced here
US20110119792A1-20110519-T00003
Please refer to the end of the specification for access instructions.

Lengthy table referenced here
US20110119792A1-20110519-T00004
Please refer to the end of the specification for access instructions.

Lengthy table referenced here
US20110119792A1-20110519-T00005
Please refer to the end of the specification for access instructions.

Lengthy table referenced here
US20110119792A1-20110519-T00006
Please refer to the end of the specification for access instructions.

Lengthy table referenced here
US20110119792A1-20110519-T00007
Please refer to the end of the specification for access instructions.

Lengthy table referenced here
US20110119792A1-20110519-T00008
Please refer to the end of the specification for access instructions.

Lengthy table referenced here
US20110119792A1-20110519-T00009
Please refer to the end of the specification for access instructions.

Lengthy table referenced here
US20110119792A1-20110519-T00010
Please refer to the end of the specification for access instructions.

Lengthy table referenced here
US20110119792A1-20110519-T00011
Please refer to the end of the specification for access instructions.

Lengthy table referenced here
US20110119792A1-20110519-T00012
Please refer to the end of the specification for access instructions.

Lengthy table referenced here
US20110119792A1-20110519-T00013
Please refer to the end of the specification for access instructions.

Lengthy table referenced here
US20110119792A1-20110519-T00014
Please refer to the end of the specification for access instructions.

Lengthy table referenced here
US20110119792A1-20110519-T00015
Please refer to the end of the specification for access instructions.

Lengthy table referenced here
US20110119792A1-20110519-T00016
Please refer to the end of the specification for access instructions.

Lengthy table referenced here
US20110119792A1-20110519-T00017
Please refer to the end of the specification for access instructions.

Lengthy table referenced here
US20110119792A1-20110519-T00018
Please refer to the end of the specification for access instructions.

Lengthy table referenced here
US20110119792A1-20110519-T00019
Please refer to the end of the specification for access instructions.

Lengthy table referenced here
US20110119792A1-20110519-T00020
Please refer to the end of the specification for access instructions.

Lengthy table referenced here
US20110119792A1-20110519-T00021
Please refer to the end of the specification for access instructions.

Lengthy table referenced here
US20110119792A1-20110519-T00022
Please refer to the end of the specification for access instructions.

LENGTHY TABLES
The patent application contains a lengthy table section. A copy of the table is available in electronic form from the USPTO web site (http://seqdata.uspto.gov/?pageRequest=docDetail&DocID=US20110119792A1). An electronic copy of the table will also be available from the USPTO upon request and payment of the fee set forth in 37 CFR 1.19(b)(3).

We claim:

1. A method for generating a transgenic plant from a host plant, said transgenic plant being more tolerant to drought condition when compared to the host plant, said method comprising the steps of:
(a) introducing into a plant cell a construct comprising a Root Specific Drought Response Gene (RSDRG) or a fragment thereof, said RSDRG or fragment thereof encoding a polypeptide; and
(b) generating a transgenic plant expressing said polypeptide.

2. The method of claim 1, wherein said RSDRG or fragment thereof is derived from a plant that is genetically different from the host plant.

3. The method of claim 1, wherein said RSDRG or fragment thereof is derived from the host plant.

4. The method of claim 1, wherein said RSDRG or fragment thereof is derived from a plant that belongs to the same species as the host plant.

5. The method of claim 1, wherein the RSDRG or fragment thereof comprises a sequence selected from the group consisting of the sequences from SEQ ID. No. 1 to SEQ ID. No. 510.

6. The method of claim 1, wherein the coding sequence of said RSDRG or fragment thereof is operably linked to a promoter for regulating expression of said polypeptide.

7. The method of claim 6, wherein the promoter is derived from another gene that is different from said RSDRG.

8. A method for generating a transgenic plant from a host plant, said transgenic plant being more tolerant to drought condition when compared to the host plant, said method comprising the steps of:
(a) introducing into a plant cell a construct comprising a DNA sequence encoding a first polypeptide that is at least 90% identical to a second polypeptide encoded by a Root Specific Drought Response Gene (RSDRG); and
(b) generating a transgenic plant expressing said first polypeptide.

9. The method of claim 8, wherein the RSDRG comprises a sequence selected from the group consisting of the sequences from SEQ ID. No. 1 to SEQ ID. No. 510.

10. The method of claim 8, wherein the coding region of said DNA sequence is operably linked to a promoter for regulating expression of said first polypeptide.

11. The method of claim 10, wherein the promoter is at least one member selected from the group consisting of a cell-specific promoter, a tissue specific promoter, an organ specific promoter, a constitutive promoter, and an inducible promoter.

12. The method according to claim 11, wherein at least a portion of said DNA sequence is oriented in an antisense direction relative to said promoter within said construct.

13. The method of claim 8, wherein the first polypeptide is at least 95% identical to said second polypeptide encoded by said RSDRG.

14. The method of claim 8, wherein the first polypeptide is at least 99% identical to said second polypeptide encoded by said RSDRG.

15. A method for generating a transgenic plant from a host plant, said transgenic plant being more tolerant to drought condition when compared to the host plant, said method comprising the steps of:
(a) introducing into a plant cell a construct comprising a DNA sequence selected from the group consisting of a soybean Root Specific Drought Response Gene (RSDRG) or a fragment thereof, and a homolog of a soybean Root Specific Drought Response Gene (RSDRG) or a fragment thereof; and
(b) generating a transgenic plant expressing a polypeptide encoded by said DNA sequence.

16. The method of claim 15, wherein the construct comprises a homolog of a soybean Root Specific Drought Response Gene (RSDRG) or a fragment thereof.

17. The method of claim 16, wherein the homolog is derived from a plant other than soybean, said homolog encoding a first polypeptide that is at least 90% identical to a second polypeptide encoded by said soybean Root Specific Drought Response Gene (RSDRG).

18. The method of claim 16, wherein the host plant is selected from the group consisting of soybean, corn, wheat, rice, and cotton.

19. A method for generating a transgenic plant from a host plant, said transgenic plant being more tolerant to drought condition when compared to the host plant, said method comprising a step of altering the expression levels of a protein encoded by a Root Specific Drought Response Gene (RSDRG) or a fragment thereof, said RSDRG being endogenous to the host plant.

20. The method of claim 19, wherein the expression levels of the protein are altered by modifying the transcription regulation of the RSDRG.

21. A transgenic plant generated from a host plant using the method of claim 1, claim 8, claim 15 or claim 19, said transgenic plant exhibiting increased tolerance to drought condition as compared to the host plant.

21. A transgenic plant generated from a host plant, said transgenic plant comprising a transgene, wherein the transgene comprises a Root Specific Drought Response Gene (RSDRG) or a fragment thereof, and said RSDRG or a fragment thereof is derived from a plant that is genetically different from the host plant.

22. The transgenic plant of claim 21, wherein the transgene comprises a sequence selected from the group consisting of sequences from SEQ ID. No. 1 to SEQ ID. No. 510.

23. The transgenic plant of claim 21, wherein the coding regions of the transgene is operably linked to a promoter for regulating expression of said transgene.

24. The transgenic plant of claim 23, wherein the promoter is at least one member selected from the group consisting of a cell-specific promoter, a tissue specific promoter, an organ specific promoter, a constitutive promoter, and an inducible promoter.

25. A transgenic plant generated from a host plant, said transgenic plant comprising a Root Specific Drought Response Gene (RSDRG) or a fragment thereof encoding a polypeptide, wherein the RSDRG is derived from a plant that belongs to the same species as the host plant, and the expression levels the polypeptide encoded by said RSDRG is altered such that the expression levels of said polypeptide in the transgenic plant is at least 50% higher or lower than the expression levels of said polypeptide in the host plant.

26. The transgenic plant of claim 25, wherein the host plant is selected from the group consisting of soybean, corn, wheat, rice, and cotton.
